(19) United States
(12) Patent Application Publication
(10) Pub. No.: US 2018/0318148 A1
(43) Pub. Date: Nov. 8, 2018

(54) Feminine Care Absorbent Article with Color Changing Indicator Graphic

(71) Applicant: Kimberly-Clark Worldwide, Inc., Neenah, WI (US)

(72) Inventors: Vishal Y. Joshi, Medellin (CO); John J. Garcia, Envigado (CO); Xuedong Song, Alpharetta, GA (US); Jose A. Peredo, Envigado (CO); Chung-Hsin Chen, Hsin Wu Hsiang (TW)

(21) Appl. No.: 15/773,082

(22) PCT Filed: Nov. 30, 2015
(86) PCT No.: PCT/US15/62967

§ 371 Date: May 2, 2018

(51) Int. Cl. A61F 13/42; A61F 13/84; A61L 15/20

(52) U.S. Cl. CPC A61F 13/42; A61F 13/84; A61F 2013/8497; A61F 2013/427; A61F 2013/422; A61L 15/20

(57) ABSTRACT

A feminine care absorbent article (e.g., feminine care pad, sanitary napkin, incontinence article, pantiliner, etc.) that employs a color changing indicator graphic for signaling to the wearer an appropriate time to remove and/or replace the feminine care absorbent article following a liquid insult. This can be particularly useful in warm environments in which a wearer may secrete a significant amount of perspiration which may act as a carrier for microorganisms. In this manner, the wearer can wear the article without fear of a bacterial infection and/or the generation of an odor. After a time period of usage, the wearer can visually inspect the color of the color changing indicator graphic for an indication of whether it is time to remove and/or replace the feminine care absorbent article.

### BACKGROUND OF THE DISCLOSURE

Feminine care absorbent articles, such as feminine care pads, sanitary napkins, pantiliners, and incontinence pads, are devices that are typically worn in the crotch region of an undergarment. Such absorbent articles are worn by women in a pair of panties and positioned between the wearer's legs adjacent to the perineum area. Such absorbent articles are designed to absorb and retain body fluids or discharges (e.g., menses) from the body of the woman and to prevent any soiling of her body and clothing.

The female vagina is naturally colonized by a variety of bacteria, yeast, and microorganisms. For example, a normal vagina generally contains more than about 10⁴ lactobacilli per milliliter of vaginal material. Under normal conditions, the vaginal flora provides a mildly acidic environment that helps guard against the invasion of pathogenic microbes (e.g., Gardnerella vaginalis, Candida albicans, etc.). Unfortunately, this vaginal balance may be easily upset by a variety of external factors that ultimately lead to a vaginal bacterial infection and/or the generation of an odor. One such external factor may be the climate. For example, in tropical climates, the consistently warm conditions can lead to a high degree of sweating near the vaginal region. An increase in moisture content due to sweating may stimulate the growth of pathogenic microorganisms which may potentially lead to bacterial infection and/or generation of odor. This problem may be particularly acute during a female's menstrual cycle when feminine care absorbent articles are used. More particularly, in addition to absorbing menstrual fluids the feminine care absorbent articles may also retain moisture secreted by the user due to sweating.

As such, a need currently exists for a feminine care absorbent article that is well suited for warm climates and that can provide a signal to the wearer that the feminine care absorbent article is soiled by a liquid insult (body exudate and/or body sweat) and should be removed and/or replaced to prevent a bacterial infection and/or the generation of an odor.

### SUMMARY OF THE DISCLOSURE

In various embodiments, an absorbent article for usage in absorbing liquid insult is characterized by comprising a topsheet layer, a backsheet layer, an absorbent core layer positioned between the topsheet layer and the backsheet layer, an intake layer positioned between the absorbent core and the topsheet layer, a color changing indicator graphic having a pre-use color and disposed on the intake layer, and a passive graphic having a pre-use color and printed on at least one of the topsheet layer, the backsheet layer, the absorbent core layer, and the intake layer, wherein the pre-use color of the color changing indicator graphic is not the same as the pre-use color of the passive graphic and the color changing indicator graphic changes to the color of the passive graphic following contact with the liquid insult. In various embodiments, the color changing indicator graphic comprises a matrix forming component, a colorant, a surfactant, and a pH adjuster, wherein the pH adjuster includes a low molecular weight organic acid and a high molecular weight organic acid. In various embodiments, the color changing indicator graphic is insoluble in the liquid insult. In various embodiments, the colorant is a pH indicator. In various embodiments, the pH indicator is selected from bromocresol green, bromophenol blue, bromochlorophenol blue, methyl orange, tetrabromophenol blue, ethyl orange, Congo red, methyl red, and allure red AR. In various embodiments, the color changing indicator graphic has an initial pH of less than 4.5. In various embodiments, the activation time of the color changing indicator graphic is from 3 to 6 hours post insult of the absorbent article with the liquid insult. In various embodiments, no adhesive material is positioned between the topsheet layer and the color changing indicator graphic.

In various embodiments, an absorbent article for usage in absorbing liquid insult is characterized by comprising a topsheet layer, a backsheet layer, an absorbent core layer positioned between the topsheet layer and the backsheet layer, an intake layer positioned between the absorbent core and the topsheet layer, a color changing indicator graphic having a pre-use color and disposed on the intake layer, and a passive graphic having a pre-use color and printed on at least one of the topsheet layer, the backsheet layer, the absorbent core layer, and the intake layer, wherein the pre-use color of the color changing indicator graphic is the same as the pre-use color of the passive graphic and the color changing indicator graphic changes to a color that is different than the color of the passive graphic following contact with the liquid insult. In various embodiments, the color changing indicator graphic comprises a matrix forming component, a colorant, a surfactant, and a pH adjuster, wherein the pH adjuster includes a low molecular weight organic acid and a high molecular weight organic acid. In various embodiments, the color changing indicator graphic is insoluble in the liquid insult. In various embodiments, the colorant is a pH indicator. In various embodiments, the pH indicator is selected from bromocresol green, bromophenol blue, bromochlorophenol blue, methyl orange, tetrabromophenol blue, ethyl orange, Congo red, methyl red, and allure red AR. In various embodiments, the color changing indicator graphic has an initial pH of less than 4.5. In various embodiments, the activation time of the color changing indicator graphic is from 3 to 6 hours post insult of the absorbent article with the liquid insult. In various embodiments, no adhesive material is positioned between the topsheet layer and the color changing indicator graphic.

### DETAILED DESCRIPTION OF THE DISCLOSURE

Generally speaking, the present disclosure is directed towards a feminine care absorbent article (e.g., feminine care pad, sanitary napkin, incontinence article, pantiliner, etc.) that employs a color changing indicator graphic for signaling to the wearer an appropriate time to remove and/or replace the feminine care absorbent article following a liquid insult (body exudate and/or body sweat). This can be particularly useful in warm environments in which a wearer may secrete a significant amount of perspiration which may act as a carrier for microorganisms. In this manner, the wearer can wear the article without fear of a bacterial infection and/or the generation of an odor. After a time period of usage, the wearer can visually inspect the color of the color changing indicator graphic for an indication of whether it is time to remove and/or replace the feminine care absorbent article.

The feminine care absorbent article can include at least four layers, such as, a liquid impermeable backsheet layer, a liquid permeable topsheet layer, an absorbent core positioned between the topsheet layer and the backsheet layer, and a liquid permeable intake layer positioned between the absorbent core and the topsheet layer. The absorbent core can include a body facing surface positioned adjacent to the intake layer and a garment facing surface positioned adjacent to the backsheet layer. The intake layer can include a body facing surface positioned adjacent to the topsheet layer and a garment facing surface positioned adjacent to the body facing surface of the absorbent core. The topsheet layer defines a body facing surface of the absorbent article and the backsheet layer defines a garment facing surface of the absorbent article. In various embodiments, the peripheral edges of each of the topsheet layer and the backsheet layer are joined by adhesive bonding, ultrasonic bonding, or any other suitable joining method known in the art, the joined edges defining an overall sealed peripheral edge of the absorbent article. The absorbent article can take on various geometries but will generally have opposing lateral edges and opposing longitudinal edges. A color changing indicator graphic may generally be disposed in fluid communication with the intake layer in a variety of different orientations and configurations. In various embodiments, the color changing indicator graphic can be applied to the intake layer of the absorbent article where it can be easily seen by the wearer and without requiring the wearer to remove the absorbent article from her panties in order to complete the visual inspection of the color changing indicator graphic.

The topsheet layer is generally designed to contact the body of the wearer and is liquid permeable. The topsheet layer may be constructed of any woven or nonwoven material that is easily penetrated by liquid insult. Examples of suitable materials include rayon, polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. The topsheet layer can be a spunbond, hydroknit, or through air bonded card web. An example of a suitable topsheet layer is a bonded carded web made of polypropylene and polyethylene such as that used as a topsheet layer for KOTEX® pantiliners and obtainable from Sandler Corporation (Germany). U.S. Pat. No. 4,801,494 to Datta, et al. and U.S. Pat. No. 4,908,026 to Sukiennik, et al. teach various other topsheet layers which are suitable for use herein. The topsheet layer may also contain a plurality of apertures formed therethrough to permit the liquid insult to pass more readily into the absorbent article. The apertures may be randomly or uniformly arranged throughout the topsheet layer, or they may be located only in a narrow longitudinal band or strip arranged along the longitudinal axis of the absorbent article. The apertures permit rapid penetration of liquid insult down into the absorbent article. The size, shape, diameter and number of apertures may be varied to suit one's particular needs. The topsheet layer can extend over the body facing surfaces of the intake layer and the absorbent core. In various embodiments, the topsheet layer can partially or entirely surround or enclose the intake layer and the absorbent core. Alternatively, the topsheet layer and the backsheet layer can have peripheral margins that extend outwardly beyond the terminal, peripheral edges of the intake layer and the absorbent core, and the extending margins of the topsheet layer and the backsheet layer may be joined together to form the overall sealed peripheral edge of the absorbent article.

The backsheet layer is generally liquid impermeable and designed to face the inner surface of a wearer's undergarment. The backsheet layer may permit passage of air or vapor out of the absorbent article while still blocking the passage of liquids. Any liquid impermeable material may generally be utilized to form the backsheet layer. For example, a suitable material that may be utilized is a microporous polymeric film, such as polyethylene or polypropylene. In various embodiments, a polyethylene film is utilized that has a thickness in the range of about 0.2 or 0.5 mils to about 3.0 or 5.0 mils. An example of a backsheet layer material is a polyethylene film such as that used in KOTEX® pantiliners and obtainable from Pliant Corporation, Schaumburg, Ill., USA.

An absorbent core is positioned between the topsheet layer and the backsheet layer and provides capacity to absorb and retain liquid insult. The absorbent core may be formed from a variety of different materials and contain any number of desired layers. For example, the absorbent core can include one or more layers of an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In various embodiments, the absorbent web material can include a matrix of cellulosic fluff and may also include superabsorbent material. The cellulosic fluff may comprise a blend of wood pulp fluff. One type of fluff is identified with the trade designation NB 416, available from Weyerhaeuser Corp., which is a bleached, highly absorbent wood pulp containing primarily soft wood fibers. The absorbent web material may be formed into a web structure by employing various conventional methods and techniques. For example, the absorbent web structure may be formed with a dry-forming technique, an air forming technique, a wet-forming technique, a foam-forming technique, or the like, as well as combinations thereof. A coform nonwoven material may also be employed. Methods and apparatus for carrying out such techniques are well known in the art.

The absorbent article can have a liquid permeable intake layer positioned between the topsheet layer and the absorbent core. The intake layer can demonstrate more hydrophilicity than the topsheet layer for liquid insult. The intake layer may be made of a material that is capable of rapidly transferring, in the z-direction, liquid insult that is delivered to the topsheet layer. The intake layer may generally have any shape and/or size as desired. In one embodiment, the intake layer has a rectangular shape, with a length equal to or less than the overall length of the absorbent article and a width less than the width of the absorbent article. Any of a variety of different materials may be used for the intake layer. The material of the intake layer may be synthetic, cellulosic, or a combination of synthetic and cellulosic materials. For example, airlaid cellulosic tissues may be suitable for use in the intake layer. The airlaid cellulosic tissue may have a basis weight ranging from about 10, 15, 20, 25, 30, 35 or 40 grams per square meter (gsm) to about 50, 100, 150, 200, 250 or 300 gsm. The airlaid cellulosic tissue may be formed from hardwood and/or softwood fibers. The airlaid cellulosic tissue can have a fine pore structure and can provide a wicking capacity for the absorbent article.

The intake layer may be maintained in secured relation with the absorbent core by bonding at least a portion of the adjacent surfaces (i.e., the garment facing surface of the intake layer and the body facing surface of the absorbent core) to one another. A variety of bonding mechanisms known to one of skill in the art may be utilized to achieve any such secured relation. Examples of such mechanisms include, but are not limited to, the application of adhesives in a variety of patterns between the two adjacent surfaces, entangling at least portions of the materials of the body facing surface of the absorbent core with portions of the materials of the garment facing surface of the intake layer, or fusing at least portions of the garment facing surface of the intake layer to portions of the body facing surface of the absorbent core. While the intake layer may be maintained in a secured relation with the absorbent core of the absorbent article, a similar relation need not exist between the intake layer and the topsheet layer. In various embodiments, as the color changing indicator graphic is disposed on the intake layer it can be preferable that no bonding mechanism, such as, for example, an adhesive, is present between the topsheet layer and the intake layer while a bonding mechanism, such as, for example, an adhesive, is present between the intake layer and the absorbent core. It is believed that a bonding mechanism, such as, an adhesive, present between the topsheet layer and the intake layer would delay liquid insult from coming into contact with the color changing indicator graphic and, therefore, would prevent the color changing indicator graphic experiencing a change in color. The wearer, therefore, would not receive the visual indicator of when it may be time to remove and/or replace the absorbent article. In various embodiments, therefore, no adhesive material is present between the topsheet layer and the color changing indicator graphic. It is believed that a bonding mechanism, such as, for example, an adhesive, present between the intake layer and the absorbent core may assist in delaying the transfer of the liquid insult from the intake layer to the absorbent core and, therefore, the liquid insult may accumulate in the intake layer providing for an extended interaction between the liquid insult and the color changing indicator graphic. The accumulation of the liquid insult in the intake layer can allow for a threshold of volume of liquid insult to be reached that can trigger the color change of the color changing indicator graphic.

In various embodiments, the amount of time that can pass between the liquid insult of the absorbent article and the color change of the color changing indicator graphic can be at least 3 hours. In various embodiments, the amount of time that can pass between the liquid insult of the absorbent article and the color change of the color changing indicator graphic can be less than 6 hours. In various embodiments, the amount of time that can pass between the liquid insult of the absorbent article and the color change of the color changing indicator graphic can be from about 3 or 4 hours to about 5 or 6 hours. It is believed that a time frame for the color changing indicator graphic to change color of from about 3 hours to about 6 hours can provide a wearer with an optimal amount of time to wear the absorbent article in her panties without a fear of bacterial infection and/or generation of odor and without the need to quickly change from one absorbent article to the next, as may occur with a rapidly changing color changing indicator graphic, thereby avoiding an increase in the cost of using the absorbent articles.

In various embodiments, the absorbent article may also contain a transfer delay layer which can be positioned between the intake layer and the absorbent core. The transfer delay layer may contain a material that is substantially hydrophobic, such as a nonwoven web composed of polypropylene, polyethylene, polyester, etc. One example of a material suitable for the transfer delay layer can be a spunbond web composed of polypropylene, multi-lobal fibers. Further examples of suitable transfer delay layer materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay layer are described in U.S. Pat. No. 4,798,603 to Meyer, et al. and U.S. Pat. No. 5,248,309 to Serbiak, et al. To adjust performance, the transfer delay layer may also be treated with a selected amount of surfactant to increase its initial wettability. In various embodiments, the transfer delay layer can have a basis weight less than that of the other layers of the absorbent core. For example, the basis weight of the transfer delay layer can be less than about 250 grams per square meter (gsm), and in some embodiments, between about 40 gsm to about 200 gsm.

In various embodiments, the absorbent article may also include laterally extending wing portions that may be integrally connected to side regions along the intermediate portion of the absorbent article. For example, the wing portions may be separately provided members that are subsequently attached or otherwise operatively joined to the intermediate portion of the absorbent article. In other configurations, the wing portions may be unitarily formed with one or more components of the absorbent article. For example, the wing portions may be formed from a corresponding, operative extension of the material employed to form the topsheet layer. Alternatively, the wing portions may be formed from a corresponding, operative extension of the material employed to form the backsheet layer, or formed from a corresponding, operative combination of the topsheet layer and backsheet layer materials.

The absorbent article can include a color changing indicator graphic which can be disposed in fluid communication with the intake layer such that the color changing indicator graphic can be easily seen by the wearer without the need for the absorbent article to be removed from the wearer's panties. The wearer can, therefore, view the color changing indicator graphic and discern whether the color changing indicator graphic is indicating that the absorbent article should be removed and/or replaced. In various embodiments, the color changing indicator graphic can be disposed on the garment facing surface of the intake layer. In various embodiments, the color changing indicator graphic can be disposed on the body facing surface of the intake layer. The color changing indicator graphic can be disposed in fluid communication with from about 0.5% to about 10% of the total area of the selected surface of the intake layer.

The color changing indicator graphic can be applied to a surface of the intake layer in any shape or design as desired, such as, dots, ovals, triangles, squares, rectangles, flowers, butterflies, hearts, stars, spirals, double spirals, clothold curves, comu spirals, polynomial double spirals, Euler's double spirals, parametric double spirals, stripes, geometric shapes, irregular shapes, alpha-numeric characters, anthropomorphic images, pictorial representation of an animal, pictorial representation of an inanimate object, pictorial representation of an item found in nature, cartoon character, product or company logo, trademark or brand name, or combination of such shapes and designs.

The color changing indicator graphic is fluid at room temperature and can be applied to a surface of the intake layer without heating. Because the color changing indicator graphic is fluid at room temperature, it can be applied through printing or stamping either directly onto the intake layer or onto a separate material which can then be associated with the intake layer. This feature of the color changing indicator graphic makes it easier to handle during the manufacture of the absorbent article. Further, the color changing indicator graphic can form a film-like printed layer when it is applied, such as by printing, to the intake layer and dried. A benefit of the color changing indicator graphic forming a film-like printed layer on the intake layer is to afford good adherence and to prevent cracking.

The color changing indicator graphic may include an organic solvent as a vehicle for the color changing indicator graphic to be applied to the intake layer wherein the organic solvent evaporates after application. The organic solvent may be a single solvent or a mixture of solvents. Suitable solvents to carry the color changing indicator graphic include ethanol, isopropanol, acetone, methanol, acetonitrile, tetrahydrofuran, benzene and toluene. When in the form of a film-like printed layer on the intake layer, the color changing indicator graphic is wettable but insoluble in water. This wettable but insoluble feature makes the color changing indicator graphic desirable for use in absorbent articles where the color changing indicator graphic will be exposed to wetness. The wettable but insoluble feature also results in a color changing indicator graphic that is durable and that is resistant to leaching out of the film-like printed layer.

The color changing indicator graphic includes a matrix forming component. The matrix forming component may include one or more water insoluble, film-forming polymers and/or one or more ink base materials. The matrix forming component of the color changing indicator graphic forms the medium that keeps the components of the color changing indicator graphic in proximity to each other. By forming the medium, the matrix forming component enables the color changing indicator graphic to be wettable, but water insoluble, and to remain as a film-like printed layer on the intake layer as opposed to migrating/leaching away from the intake layer. The water insoluble, film-forming polymers are solid at room temperature, but soluble in a volatile organic solvent or an organic mixing solvent so that when used, the color changing indicator graphic is liquid at room temperature. The ink base materials are liquid at room temperature. When an ink base material is included in the matrix forming component, a volatile organic solvent may or may not be used. Desirably, the water insoluble, film forming polymers/copolymers have a substantial amount, greater than about 0.5% by weight, of polar atoms such as oxygen and nitrogen. The polar atoms may be present in polar functional groups such as amides, carboxylic acids and esters. Preferably, the water insoluble polymers/copolymers are soluble in a volatile organic solvent such as ethanol, acetone, methanol, acetonitrile, tetrahydrofuran, benzene, toluene and mixtures of such solvents. The water insoluble, film forming polymer and the other components of the color changing indicator graphic can be dissolved in the organic solvent prior to application onto the intake layer. When the mixture of the color changing indicator graphic and the organic solvent is formed, the mixture is liquid at room temperature. The volatile organic solvent evaporates when the color changing indicator graphic is applied to the intake layer. The color changing indicator graphic includes a matrix forming component in an amount of from 20% to 95% of the total weight of the color changing indicator graphic. In various embodiments, the color changing indicator graphic includes a matrix forming component in an amount of from 20% to 90% of the total weight of the color changing indicator graphic.

Suitable water soluble, film forming polymers include acrylate/acrylamide copolymers, polyurethane adhesives, copolymers of vinylpyrrolidone, and copolymers of dimethyl aminopropyl methacrylamide. Commercially available suitable polymers include DERMACRYL 79 polymer and AMPHOMER HC polymer, both of which are acrylate/octylacrylamide copolymers available from Akzo Nobel. Another example of a commercially available suitable polymer is GANTREZ SP polymer, which is a monoalkyl ester of poly(methyl vinyl ether/maleic acid) copolymer available from International Specialty Products Inc.

Suitable ink base materials may be small molecules, polymeric materials or a mixture of small molecules and polymers. Examples of suitable small molecule base materials include glycols, including triglycerols and their derivatives. Examples of suitable polymeric materials that may be used as ink base materials include polyvinyl alcohol, polyvinyl pyrrolidone, polyethylene glycol, water soluble derivatives of polyacrylates and polyacrylamides, poly(hydroxyethyl methacrylates), poly(hydroxylethyl acrylates), carboxymethyl cellulose, gelatin and gum Arabic. Another suitable ink base material for the color changing indicator graphic is a varnish base such as a nitrocellulose compound based varnish or a phenolic-modified co-solvent-type polyamide resin-based varnish. Further, the ink base material can be a flexographic varnish having an organic solvent base. It is believed that the ink base material may help the stability of the color changing indicator graphic. It is also believed that the ink base material may improve the adhesion of the color changing indicator graphic to the intake layer. The ink base material may be water soluble or water insoluble.

The color changing indicator graphic also includes a colorant, preferably a charged colorant, which functionally acts as a pH indicator. The colorant may be a neutral pH indicator, a charged pH indicator or a zwitterionic pH indicator. The colorant desirably changes color at either a pH greater than 9.5 or lower than 5.5. The color change may be from color to colorless, colorless to color, or from one color to another color. The charged colorant has the charged functional groups either in the core chromophore structure or derivatized in pendant groups. The colorant may be derivatized as a polymer. Examples of suitable colorants include the following: gentian violet (methyl violet), leucomalachite green, methyl yellow, bromophenol blue, Congo red, methyl orange, malachite green, brilliant green, crystal violet, erythrosine B, methyl green, methyl violet 2B, picric acid, napthol yellow S, quinaldine red, Eosin Y, basic fuchsin, 4-(p-anilinophenylazo)benzene-sulfonic acid, sodium salt, phloxine B, bromochlorophenol blue W. S., ethyl orange, bromocresol nile blue A, thymolphthalein, aniline blue W. S., alizarin yellow GG, morgant orange I, tropaeolin O, orange G, acid fuchsin, thiazol yellow G, indigo carmine, phenolphthalein, thymolphthalein, alizarine yellow R, bromocresol green and their respective derivatives. The color changing indicator graphic includes a colorant in an amount of from 0.1% to 10% of the total weight of the color changing indicator graphic. In various embodiments, the color changing indicator graphic includes a colorant in an amount of from 0.5% to 5% of the total weight of the color changing indicator graphic. The color changing indicator graphic may include more than one colorant. One or more colorants that have visually different colors may be combined or colorants having the same visual color may be combined.

The color changing indicator graphic may also include a surfactant, preferably oppositely-charged surfactant that attracts the positively or negatively charged colorant. The surfactant may also include a neutral surfactant. It is believed that the surfactant serves a dual role in the color changing indicator graphic. In one regard, the surfactant adjusts the wettability of the color changing indicator graphic so that fluids can penetrate the color changing indicator graphic to change the pH, resulting in a change of the color of the colorant. In a second regard, the surfactant component forms a charge/charge complex with a colorant so that the colorant is imbedded in the color changing indicator graphic with minimal leaching. One dimension of the performance of the color changing indicator graphic is their response time. Response time can be made faster by increasing the wettability of the color changing indicator graphic (by increasing the amount of surfactant) so that fluids can penetrate more rapidly. However, a potential downside of increasing the amount of surfactant is a reduction in stability—particularly at elevated temperature and humidity conditions. Including a neutral surfactant can increase wettability without affecting the stability of the color changing indicator graphic. Therefore, using both neutral surfactants and charged surfactants in the color change indicator can result in color changing indicator graphics with improved wettability and good stability.

The surfactant may be a small molecule or a polymer. Suitable positively charged surfactants include benzathonium chloride and benzethonium chloride. Mixtures of positively charged surfactants may also be used. Suitable negatively charged surfactants include alkyl sulfates such as sodium laurylsulfate, sodium dodecylsulfate and sodium tetradodecyl sulfate. Alkylbenzenesulfates such as sodium dodecylbenzenesulfonate and sodium diheptylsulocuccinate are suitable negatively charged surfactants. Additional suitable negatively charged surfactants include dodecyltrimethyl ammonium chloride, stearateamine acetate, sodium polyoxyethylenealkyl ether sulfate and triethanolamine poly oxyethylenealkyl ether sulfate. Mixtures of negatively charged surfactants may also be used.

In addition to charged surfactants, the color changing indicator graphic may include a neutral surfactant. Suitable neutral surfactants include Tween 20, Tween 40, Tween 80, Triton-X-100, polyethylene lauryl ether, polyoxyethylene nonyl phenyl ether, polyoxyethylene oleyl phenyl ether, polyoxyethylene sorbitan monolaurate, polyethylene glycol monostearate, polyethylene glycol sorbitan monolaurate, polyoxyethylene sorbitan monopalmitate, polyoxyethylenesorbitan monostearate, polyoxyethylenesorbitan monooleate, polyoxyethylenesorbitan trioleate, polypropylene glycol sorbitan monolaurate, polyoxypropylenesorbitan monopalmitate, polyoxypropylenesorbitan monostearate, polyoxypropylenesorbitan monooleate, polyoxypropylenesorbitan trioleate, polyalkyne glycol sorbitan monolaurate, polyalkyne glycol sorbitan monopalmitate, polyalkyne glycol sorbitan monostearate, polyalkyne glycol sorbitan monooleate, polyalkyne glycol sorbitan trioleate and mixtures of such neutral surfactants.

The color changing indicator graphic can include surfactants in an amount of from 2% to 50% of the total weight of the color changing indicator graphic. In various embodiments, the color changing indicator graphic can include oppositely charged surfactants in an amount of from 10% to 30% of the total weight of the color changing indicator graphic. With regard to the relative amounts of the different types of surfactants, the ratio of charged surfactant to neutral surfactant can range from 0.2 to 10.

In addition to the other components, the color changing indicator graphic includes a pH adjuster. The pH adjuster is any molecule or composition that may be used to control the pH of the color changing indicator graphic. The pH adjuster may be an acid, a base or a combination of both such as would be found with a buffering composition. The pH adjuster is selected in conjunction with the choice of colorant to be used in the color changing indicator graphic. For example, if the color changing indicator graphic includes a colorant that has a color transition point that occurs at a pH of lower than 5.5, the selected pH adjuster is desirably an acid to make the pH of the color changing indicator graphic acidic. If the color changing indicator graphic includes a colorant that transitions color at a pH higher than 9.5, the selected pH adjuster is desirably a base to make the pH of the color changing indicator graphic basic.

Examples of suitable acid pH adjusters includes organic acids, inorganic acids, and polymeric acids: more specifically, examples of low molecular weight organic acids include glycolic acid, citric acid, lactic acid, ascorbic acid, oxalic acid, maleic acid, tartaric acid, salicylic acid, palmitic acid and stearic acid. Examples of high molecular weight organic acids include polyacrylic acid, polymethacrylic acid, and copolymers containing acrylic acids, methacrylic acids or both acrylic acids and methacrylic acids.

Both the low molecular weight acids and the high molecular weight acids can be “polymeric.” Depending on the number of monomer units, the polymeric acid will either be low molecular weight (typically less than 1000 Daltons) or high molecular weight (typically greater than 1000 Daltons). The low molecular weight organic acids having repeating monomer units may be referred to as oligomers. In some aspects, the polymeric acid can be a “bidentate” or higher order acid. By “bidentate or higher order” it is meant that the polymeric acid has more than one acid group in its smallest polymer building block. This can be easily understood when one compares ascorbic acid to tartronic acid (two acid groups) and citric acid (three acid groups). In some aspects, the polymeric acids may be a dendrimer or the like where the dendrimer's surface and interior are fully functionalized with acid groups. Examples of suitable, simple polymeric acids for which the number of repeating monomer units can vary are salicylic acid and ascorbic acid; if the molecular weight is less than 1000 Daltons, the acid may be referred to as an oligomer and if the molecular weight is greater than 1000 Daltons, the acid may be referred to as a polymer. The following are examples of suitable dicarboxylic polymeric acids:

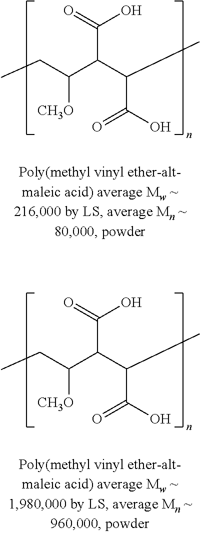

Examples of suitable tricarboxylic polymeric acids include the following:

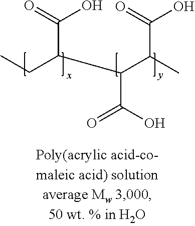

Examples of suitable polyacrylic acids include the following: polyacrylic acid having an average molecular weight of about 1800 Daltons, polyacrylic acid having an average molecular weight of about 450,000 Daltons, polyacrylic acid having an average molecular weight of about 1,250,000 Daltons and polyacrylic acid having an average molecular weight of about 3,000,000 Daltons. An example of a suitable, strong polymeric acid is poly(vinylphosphonic acid).

Examples of suitable basic pH adjusters include organic bases, inorganic bases and polymeric bases; more specifically, examples include sodium hydroxide, sodium carbonate, sodium bicarbonate, sodium borate, potassium hydroxide, polymeric amines, dendrimeric amine and 1,3-pentanediamine. Combination pH adjusters that have a buffering effect include acetic buffer, borate buffer and carbonate buffer. In various embodiments, the pH of the combination pH adjuster is either greater than 10 or lower than 5. Typically, the combination pH adjuster is in solution form and the concentration of the buffer may range from about 0.01 milliMolar to about 1000 milliMolar and desirably range from about 1 milliMolar to about 20 milliMolar, depending on the combination pH adjuster selected. The color changing indicator graphic can include a pH adjuster in an amount of from 0.1% to 20% of the total weight of the color changing indicator graphic. In various embodiments, the color changing indicator graphic can include a pH adjuster in an amount of from 0.5 to 5% of the total weight of the color changing indicator graphic. With respect to the relative amounts of organic acid, the ratio of low molecular weight organic acid to high molecular weight organic acid can range from 0.02 to 50.

Benefits of the pH adjuster include stabilizing the colorant against premature color changes that may be caused by exposure to humid environments. For example, the pH adjuster is believed to maintain a stable pH, such as a low pH environment with an acidic pH adjuster, around the colorant even when the color changing indicator graphic is exposed to high humidity.

An example of a color changing indicator graphic is a composition that includes an acrylate and octylacrylamide copolymer system as the water-insoluble, film-forming polymer. This polymer can assist adhesion of the colorant to the layer of the absorbent article and provide wettability with water resistance. Another example of a color changing indicator graphic is a composition that includes a polyurethane adhesive with amide functionality as the water-insoluble, film-forming polymer. This water-insoluble, film-forming polymer can be dissolved in an alcohol for application to the intake layer (with the alcohol subsequently evaporating).

The amount of color changing indicator graphic present on the intake layer can range from about 0.04 gsm to about 3.5 gsm. The color changing indicator graphic can, therefore, provide a non-tactile signal within the feminine care absorbent article to the wearer in response to a liquid insult thereof. The color changing indicator graphic can, therefore, be considered an “active” graphic. An “active graphic” refers to a graphic that visibly (to an unaided human eye) changes appearance in response to a liquid insult of the feminine care absorbent article. For example and as described herein, the color changing indicator graphic changes color from a first color to a second color in response to a liquid insult.

In addition to the color changing indicator graphic, the feminine care absorbent article can include a passive graphic disposed on a layer of the absorbent article such that the passive graphic can be easily seen by the wearer without the need for the absorbent article to be removed from the wearer's panties. A “passive graphic” refers to a graphic which is inactive, or permanent, and does not change in visible appearance in response to a liquid insult to the feminine care absorbent article. The passive graphic can include a graphic in any shape or design as desired, such as, dots, ovals, triangles, squares, rectangles, flowers, butterflies, hearts, stars, spirals, double spirals, clothold curves, comu spirals, polynomial double spirals, Euler's double spirals, parametric double spirals, stripes, geometric shapes, irregular shapes, alpha-numeric characters, anthropomorphic images, pictorial representation of an animal, pictorial representation of an inanimate object, pictorial representation of an item found in nature, cartoon character, product or company logo, trademark or brand name, or combination of such shapes and designs. The passive graphic can be applied to a layer of the absorbent article in any manner deemed suitable such as by printing or spraying the passive graphic onto the desired layer of the absorbent article. In various embodiments, the passive graphic can be disposed on the same layer of the absorbent article as the color changing indicator graphic. In various embodiments, the passive graphic can be disposed on a different layer of the absorbent article than the color changing indicator graphic.

In use, the color changing indicator graphic, the active graphic, can act in concert with the passive graphic to provide an indication to the wearer of the feminine care absorbent article as to whether the feminine care absorbent article needs to be removed and/or replaced. Together, the color changing indicator graphic and the passive graphic can impart a “pre-use” visual appearance as well as can impart a “post-use” visual appearance.

In various embodiments, each of the color changing indicator graphic and the passive graphic can have a pre-use color and can be applied to the same or different layer of a feminine care absorbent article. In such embodiments, the pre-use color of each of the color changing indicator graphic and the passive graphic can be different from each other. In such embodiments, during usage of the absorbent article, a liquid insult to the absorbent article can interact with the color changing indicator graphic which can result in an alteration of the pH of the colorant of the color changing indicator graphic further resulting in a color change such that the color of the color changing indicator graphic changes from the pre-use color to a post-use color. The pre-use color of the passive graphic does not change and, therefore, the post-use color of the passive graphic is the same as the pre-use color of the passive graphic. In such embodiments, the post-use color of the color changing indicator graphic is the same as the post-use color of the passive graphic. As an example of such embodiments, the color changing indicator graphic can include a charged colorant which can functionally act as a pH indicator and the color changing indicator graphic can, therefore, have an initial pre-use pH value. The pre-use color of the color changing indicator graphic and the pre-use color of the passive graphic can be different from each other. During usage, a liquid insult to the absorbent article can interact with the color changing indicator graphic which can result in an alteration of the pH of the colorant of the color changing indicator graphic. This alteration in the pH can cause the colorant to change color from the pre-use color to the post-use color which, in this example, is the same as the post-use color of the passive graphic. In such embodiments, the pre-use visual appearance of the absorbent article is that the absorbent article has graphics printed in two colors and the post-use visual appearance of the absorbent article is that the absorbent article has graphics printed in a single color. In such embodiments, the presence of only a single color for the graphics is the signal to the wearer that the absorbent article should be removed and/or replaced.

In various embodiments, each of the color changing indicator graphic and the passive graphic can have a pre-use color and can be applied to the same or different layer of a feminine care absorbent article. In such embodiments, the pre-use color of each of the color changing indicator graphic and the passive graphic can be the same as each other. In such embodiments, during usage of the absorbent article, a liquid insult to the absorbent article can interact with the color changing indicator graphic which can result in an alteration of the pH of the colorant of the color changing indicator graphic further resulting in a color change such that the color of the color changing indicator graphic changes from the pre-use color to a post-use color. The pre-use color of the passive graphic does not change and, therefore, the post-use color of the passive graphic is the same as the pre-use color of the passive graphic. In such embodiments, the post-use color of the color changing indicator graphic is different from the post-use color of the passive graphic. As an example of such embodiments, the color changing indicator graphic can include a charged colorant which can functionally act as a pH indicator and the color changing indicator graphic can, therefore, have an initial pre-use pH value. The pre-use color of the color changing indicator graphic and the pre-use color of the passive graphic can be the same as each other. During usage, a liquid insult to the absorbent article can interact with the color changing indicator graphic which can result in an alteration of the pH of the colorant of the color changing indicator graphic. This alteration in the pH can cause the colorant to change color from the pre-use color to the post-use color which, in this example, is different from the post-use color of the passive graphic. In such embodiments, the pre-use visual appearance of the absorbent article is that the absorbent article has graphics printed in a single color and the post-use visual appearance of the absorbent article is that the absorbent article has graphics printed in two colors. In such embodiments, the presence of multiple colors for the graphics is the signal to the wearer that the absorbent article should be removed and/or replaced.

A first example of a color changing indicator graphic is as follows:

A color changing indicator graphic includes a matrix forming component of a flexographic varnish having an organic solvent base in an amount of 2.5 ml. The color changing indicator graphic also includes a colorant of bromocresol green in an amount of 15 mg/ml and a surfactant of benzethenium chloride in an amount of 20 mg/ml. The color changing indicator graphic includes a pH adjuster of a low molecular weight organic acid, citric acid, in an amount of 200 mg/ml and high molecular weight organic acid, polyacrylic acid, in an amount of 200 mg/ml. The colorant, surfactant and pH adjusters are combined together and dissolved in 3 ml of ethanol. This solution is then mixed with the matrix-forming component by vortex until a homogenous solution is formed.

A second example of a color changing indicator graphic is as follows:

A color changing indicator graphic includes a matrix forming component of a flexographic varnish having an organic solvent base in an amount of 2.5 ml. The color changing indicator graphic includes a colorant of bromocresol green in an amount of 15 mg/ml. The composition also includes a surfactant of benzethenium chloride in an amount of 150 mg/ml and a neutral surfactant of Tween 40 in an amount of 50 mg/ml. The color changing indicator graphic includes a pH adjuster of a low molecular weight organic acid, citric acid, in an amount of 200 mg/ml and a high molecular weight organic acid, polyacrylic acid, in an amount of 200 mg/ml. The colorant, surfactants and pH adjusters are combined together and dissolved in 3 ml of ethanol. This solution is then mixed with the matrix-forming component by vortex until a homogenous solution is formed.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article for usage in absorbing liquid insult characterized by comprising a topsheet layer, a backsheet layer, an absorbent core layer positioned between the topsheet layer and the backsheet layer, an intake layer positioned between the absorbent core and the topsheet layer, a color changing indicator graphic having a pre-use color and disposed on the intake layer, and a passive graphic having a pre-use color and printed on at least one of the topsheet layer, the backsheet layer, the absorbent core layer, and the intake layer, wherein the pre-use color of the color changing indicator graphic is not the same as the pre-use color of the passive graphic and the color changing indicator graphic changes to the color of the passive graphic following contact with the liquid insult.

2. The absorbent article of claim 1 wherein the color changing indicator graphic comprises a matrix forming component, a colorant, a surfactant, and a pH adjuster, wherein the pH adjuster includes a low molecular weight organic acid and a high molecular weight organic acid.

3. The absorbent article of claim 1 wherein the color changing indicator graphic is insoluble in the liquid insult.

4. The absorbent article of claim 1 wherein the colorant is a pH indicator.

5. The absorbent article of claim 4 wherein the pH indicator is selected from bromocresol green, bromophenol blue, bromochlorophenol blue, methyl orange, tetrabromophenol blue, ethyl orange, Congo red, methyl red, and allure red AR.

6. The absorbent article of claim 1 wherein the color changing indicator graphic has an initial pH of less than 4.5.

7. The absorbent article of claim 1 wherein the activation time of the color changing indicator graphic is from 3 to 6 hours post insult of the absorbent article with the liquid insult.

8. The absorbent article of claim 1 wherein no adhesive material is positioned between the topsheet layer and the color changing indicator graphic.

9. An absorbent article for usage in absorbing liquid insult characterized by comprising a topsheet layer, a backsheet layer, an absorbent core layer positioned between the topsheet layer and the backsheet layer, an intake layer positioned between the absorbent core and the topsheet layer, a color changing indicator graphic having a pre-use color and disposed on the intake layer, and a passive graphic having a pre-use color and printed on at least one of the topsheet layer, the backsheet layer, the absorbent core layer, and the intake layer, wherein the pre-use color of the color changing indicator graphic is the same as the pre-use color of the passive graphic and the color changing indicator graphic changes to a color that is different than the color of the passive graphic following contact with the liquid insult.

10. The absorbent article of claim 9 wherein the color changing indicator graphic comprises a matrix forming component, a colorant, a surfactant, and a pH adjuster, wherein the pH adjuster includes a low molecular weight organic acid and a high molecular weight organic acid.

11. The absorbent article of claim 9 wherein the color changing indicator graphic is insoluble in the liquid insult.

12. The absorbent article of claim 9 wherein the colorant is a pH indicator.

13. The absorbent article of claim 12 wherein the pH indicator is selected from bromocresol green, bromophenol blue, bromochlorophenol blue, methyl orange, tetrabromophenol blue, ethyl orange, Congo red, methyl red, and allure red AR.

14. The absorbent article of claim 9 wherein the color changing indicator graphic has an initial pH of less than 4.5.

15. The absorbent article of claim 9 wherein the activation time of the color changing indicator graphic is from 3 to 6 hours post insult of the absorbent article with the liquid insult.

16. The absorbent article of claim 9 wherein no adhesive material is positioned between the topsheet layer and the color changing indicator graphic.
